(19) United States
(12) Patent Application Publication
(10) Pub. No.: US 2005/0215639 A1
(43) Pub. Date: Sep. 29, 2005

(54) Disinfectant with activity against hepatitis b virus

(76) Inventors: Michael Mohr, Kaltenkirchen (DE); Peter Goroncy-Bermes, Hamburg (DE)

Correspondence Address: Air Liquide;Intellectual Property Department, Suite 1800, 2700 Post Oak Boulevard, Houston, TX, 77056, US

(21) Appl. No.: 10/515,258

(22) PCT Filed: Mar. 28, 2003
(86) PCT No.: PCT/IB03/01137

§ 371 Date: Nov. 19, 2004

(30) Foreign Application Priority Data
May 29, 2002 (DE) 102 23 934.7

(51) Int. Cl. A61K 31/202; A61K 31/14

(52) U.S. Cl. 514/560; 514/643; 514/642

(57) ABSTRACT

A composition and a method of using the composition as a disinfectant for inactivating the hepatitis B virus. The composition is contains at least one alkylamine and/or at least one quaternary ammonium compound, and at least one fatty acid RCOOH and/or a salt thereof, where R is a group having at least 7 carbon atoms.

### BACKGROUND

The invention relates to disinfectants and to the use thereof for inactivating hepatitis B virus.

Three groups of substances have been used to date in particular for the inactivation of viruses such as hepatitis B virus using surface (skin, floor) and instrument disinfectants:
short-chain organic acids such as formic acid, acetic acid, citric acid.
The effect of such monobasic or polybasic acids is disclosed inter alia in EP-A-0 505 763 and AT-A 3823190, see also Hygiene+Medizin 1989, 14, pages 69 et seq., GB-A-2 103 089 and Tierärztliche Umschau 1988, 43, pages 646 et seq. However, a disadvantage which has emerged is that such disinfectants inevitably have a low pH and accordingly have a strong corrosive action especially when used at elevated temperatures for disinfecting instruments.
Quaternary ammonium compounds.
These have proved to be, especially in disinfectants with a very high alcohol content, e.g. in anhydrous isopropanol/n-propanol or 80% ethanol, effective disinfectants for the hands (see, inter alia, Wallhäuser, Praxis der Sterilisation, Henkel Chemische Bibliothek, 4th edition, 1988, pages 75 et seq.). However, tests by the Applicant have shown that quaternary ammonium compounds are not reliably effective for HBV in solutions with very low alcohol content. On the other hand, disinfectants with high alcohol concentrations are unsuitable inter alia for disinfecting instruments because they attack plastic materials. In addition, disinfectants containing quaternary ammonium compounds are high-foaming, which restricts their use, especially for disinfecting instruments.
Aldehydes such as formaldehyde, acetaldehyde and glutaraldehyde.
Aldehyde-containing products show excellent activity against HBV. However, aldehyde-containing disinfectants have been unwanted for some years because of the harmful effects on human health, especially of formalin.

### SUMMARY

Accordingly, the object on which the present invention was based was to provide disinfectants which
1. for reasons of cost and for good tolerability on possible contact of the user with the disinfectant are low in active ingredient and well tolerated (non-irritant) for people,
2. are not inevitably strongly acidic and corrosive,
3. have no foaming action,
4. can be formulated with low alcohol content,
5. do note inevitably require the presence of aldehyde,
6. are compatible with other optional ingredients and
7. inactivate hepatitis B virus outstandingly even used in low concentration.

The proposal according to the invention for the inactivation of HBV is a water-based disinfectant which comprises
a) at least one alkylamine and/or at least one quaternary ammonium compound and
b) at least one fatty acid RCOOH and/or salt thereof, where R is a group having at least 7 carbon atoms.

### DESCRIPTION OF PREFERRED EMBODIMENTS

Examples of quaternary ammonium compounds (quats) and alkylamines which can be used according to the invention are benzalkonium chloride, didecyldimethylammonium chloride and dioctyldimethylammonium chloride, and bis(3-aminopropyl)octylamine, aminopropyldodecylamine, dodecylpropylenediamine, coconut fatty amine 2EO and dimethyl coconut fatty amine. It is possible to employ quat mixtures, amine mixtures and also mixtures of quat(s) and amine(s). A mixture of dioctyldimethylammonium chloride with bis(3-aminopropyl)octylamine is particularly preferred.

Disinfectants which have proved to be particularly effective comprise from 1 to 40% by weight, preferably 3 to 25% by weight, in particular 5 to 20% by weight, of alkylamine and/or quaternary ammonium compound, based on the disinfectant.

In a disinfectant according to the invention, the group R of the fatty acid is preferably saturated or unsaturated, unbranched or branched, unsubstituted or substituted C₉- to C₂₅-alkyl, preferably C₁₂- to C₂₃-alkyl. R is particularly preferably monounsaturated C₁₃- to C₂₁-alkyl, e.g. RCOOH is oleic acid.

The skilled person is aware that fatty acids and their salts are partly dissociated when present in aqueous solutions. Salts of the fatty acids are preferably employed to formulate the disinfectants employed according to the invention, such as alkali metal or ammonium salts, in particular sodium salts, for example sodium oleate.

The content of component b) is preferably from 0.05 to 5% by weight, in particular 0.1 to 3% by weight, such as 0.5 to 2% by weight, based on the disinfectant (calculated as free acid RCOOH). In a particularly preferred embodiment, the disinfectant comprises from 5 to 20% by weight of quaternary ammonium compound and/or alkylamine and from 0.5 to 2% by weight of sodium oleate.

Besides the components which are stipulated as obligatory according to the invention, the disinfectant used according to the invention additionally comprises where appropriate one or more other substances such as nonionic surfactants, short-chain organic acids such as lactic acid, glycolic acid, citric acid, malic acid, succinic acid, tartaric acid, formic acid, acetic acid, propionic acid or salts thereof, corrosion inhibitors, perfume, dye and alcohols. The short-chain organic acids are employed in particular for adjusting the amine formulations to the preferred pH of from 9.0 to 9.5. In the case of quat formulations, where appropriate adjustment to the desired pH of from 9.0 to 9.5 is necessary with basifying substances. This is possible for example with sodium hydroxide solution, but N,N,N′,N′-tetrakis(2-hydroxypropyl)ethylenediamine is particularly suitable. The content of every single one of the other substances is preferably up to 5% by weight.

A preferred alcohol is isopropanol. It is also possible to employ in addition or in place of isopropanol other alcohols such as ethanol or n-propanol or glycols or aromatic alcohols such as phenoxypropanols, which act as solubilizers to stabilize the concentrate. The alcohols prevent crystallization, improve the low-temperature stability and serve as further active ingredients; their concentration can also be distinctly higher than 5% by weight and be, for example, from 10 to 50% by weight, such as 20 to 40% by weight.

In a particularly preferred embodiment, the disinfectant displays as 2% by weight aqueous solution a foaming power of less than 45 ml, preferably less than 42 ml, in particular less than 39 ml or even 37 ml or less, determined by the following method:
1. foam-free introduction of 30 ml of solution into a 100 ml measuring cylinder at 20 to 25° C.,
2. placing of a stopper on the measuring cylinder,
3. vigorous shaking ten times and
4. after the end of the shaking immediate determination of the height of the foam, stated as volume of the solution including foam.

The invention further relates to the use of the disinfectant for disinfecting surfaces and instruments. Surfaces are generally disinfected by scouring or wiping methods. Instruments are disinfected by manual insertion of the instruments or by mechanical methods in automatic processors. Typically employed in the said method is a ready-to-use solution which represents an aqueous solution of the disinfectant and comprises from 0.3 to 10% by weight, preferably 0.5 to 5% by weight, in particular 1 to 3% by weight, e.g. 2% by weight, of the disinfectant.

Thermochemical disinfection of instruments, especially temperature-sensitive instruments such as flexible endoscopes, is carried out in special automatic cleaning and disinfection systems. An example of a program flow in which the disinfectant according to the invention can be employed advantageously in the form of an instrument disinfectant is as follows:
1. where appropriate precleaning with cold water,
2. cleaning at 55 to 60° C. with a neutral cleaner (e.g. as 0.5% strength solution),
3. thermochemical disinfection at 55 to 60° C. and an exposure time of from 1 to 20 minutes, e.g. about 5 minutes, to a disinfectant according to the invention (e.g. 1 to 3% strength),
4. rinsing with cold water and
5. drying.

For thermochemical disinfection, the three parameters of concentration, exposure time and temperature are chosen suitably by the skilled person. Particularly preferred disinfectants contain the following components in the following quantities:

(i) Surface Disinfectant (Data in % by Weight)

| | preferred | especially |
|---|---|---|
| Quaternary ammonium compound, in | 5-10 | 6-9 |
| particular dioctyldimethylammonium | | |
| chloride | | |
| Arom. alcohol | no | |
| Fatty acid, in particular oleic acid, | 0.3-3 | 0.8-1.6 |
| as sodium salt | | |
| Nonionic surfactant | 0.2-1 | 0.3-0.7 |
| Alcohol | no | |
| Amine (pH adjustment) | no | |
| Corrosion inhibitor | yes | |
| Perfume | yes | |
| Dye | yes | |

(ii) Instrument Disinfectant (Data in Percent by Weight)

| | preferred | especially |
|---|---|---|
| Quaternary ammonium compound, in | 10-20 | 13-17 |
| particular dioctyldimethylammonium | | |
| chloride | | |
| Arom. alcohol | 1-10 | 3-7 |
| Fatty acid, in particular oleic acid, | 1-7 | 3-5 |
| as sodium salt | | |
| Nonionic surfactant | 1-3 | 1.5-2.5 |
| Alcohol | 10-50 | 20-40 |
| Amine (pH adjustment) | yes | |
| Corrosion inhibitor | yes | |
| Perfume | no | |
| Dye | no | |

The advantages of the invention are evident in particular from the following examples. Unless stated otherwise, all percentage data in the examples are based on weight.

### EXAMPLES

The following substances were used:
Bardac LF: dioctydimethylammonium chloride (50% strength),
Lonzabac LF: bis(3-aminopropyl)octylamine and
nonionic surfactant: alkylpolyoxylakylene glycol ether (low-foaming surfactant).

### Formulations Based on Quaternary Ammonium Compound

| | 1A | 1B |
|---|---|---|
| Bardac LF | 15% | 15% |
| Sodium oleate | | 1% |
| Sodium citrate | 5% | |
| Nonionic surfactant | 2% | 2% |
| Water | 78% | 82% |

### Formulations Based on Alkylamine

| | 2A | 2B |
|---|---|---|
| Lonzabac LF | 7.5% | 7.5% |
| Sodium oleate | | 1.5% |
| Nonionic surfactant | 4% | |
| Malic acid | 3.5% | 3.5% |
| Corrosion inhibitor | 0.1% | 0.1% |
| Isopropanol | | 5% |
| Water | 84.9% | 78.4% |

Investigation of the HBV Activity of Formulations

For the investigations with the formulations, the destruction of the antigenicity of the surface antigen (hepatitis B surface antigen=HBsAG) was used as indirect marker of hepatitis B activity. The formulations were in each case diluted with sterile double-distilled water to the desired concentrations (1% by weight, 2% by weight and 3% by weight) immediately before the inactivation tests.

The disinfectant tests took place in a suspension test at room temperature, with the volume ratios and the protein loading being carried out in accordance with the guideline of the German health agency (BGA) and the German association for controlling viral diseases (DVV) on the testing of the activity of chemical disinfectants against viruses, see Bundesgesundheits-blatt 25, 1982, pages 397-8. The test mixture consisted of one part of an HBsAg-containing serum (HBsAg and HBeAg pos., DNA polymerase detectable, HBV PCR pos., virus genomes≧10⁸/ml), one part of double-distilled water or one part of a 2% strength serum albumin solution or one part of foetal calf serum (FCS) and eight parts by volume of the formulation to be tested (disinfectant) in 1.25 times the desired concentration. Immediately after the exposure time had elapsed, the test mixture was diluted 1:100 with PBS containing 10% FCS in order to abolish the effect of the disinfectant by dilution. Each mixture was subsequently investigated as duplicate determination of HBsAg in a radioimmuno-assay (RIA) (Ausria II, Abbott Lab., North Chicago, Ill., USA) and the average radioactivity was calculated.

The following controls were also included. A test mixture containing double-distilled water in place of the disinfectant was investigated for HBsAg after the longest of the tested exposure times. This mixture, which was also carried out with serum albumin and FCS, represented the initial values for calculating the increase in HBsAg. A test mixture without added virus (disinfectant controls) and a test mixture exclusively with the diluent also took place in order to identify in this way, by comparison of the values, a nonspecificity due to disinfectant present (see attached key).

Positive and negative controls from the manufacturer's test kit were also included.

Then, in accordance with the method described by G. Frösner, G. Jentsch, H. Uthermann in Zbl. Bakt. Hyg. I. Abt. Orig. B 176, 1, (1982) “Zerstörung der Antigenität und Beeinflussung der immunochemischen Reaktivität von Antigenen des Hepatitis-B-Virus (HbₛAg, Hb_{c}Ag and HbₑAg) durch Desinfektionsmittel im Prüfmodell”, complete inactivation of HBsAg was assumed if the radioactivity (cpm) after exposure to the disinfectant was less than 2.1 times the radioactivity (cpm) of the test mixture without added virus. The disinfectant in these test mixtures was mixed with double-distilled water, serum albumin or FCS, and was then diluted 1:100 in PBS with 10% FCS in accordance with the description above.

### Explanation of the Mixtures

HBsAG Controls
I serum+double-distilled water+double-distilled water
II serum+2% albumin+double-distilled water
III serum+FCS+double-distilled water

Disinfectant Controls
I double-distilled water+double-distilled water+disinfectant
II double-distilled water+2% albumin+disinfectant
III double-distilled water+FCS+disinfectant

Diluent Control
I 10% FCS in PBS

Inactivation Mixture
I serum+double-distilled water+disinfectant
II serum+2% albumin+disinfectant
III serum+FCS+disinfectant
   Investigation of the Foaming Power of Formulations

The following tests serve to assess the foaming power of a formulation solution (disinfectant solution).

A 100 ml measuring cylinder (high form with graduation and lettering) with a fitting stopper and a stop-clock are required. For the investigation, 30 ml of the formulation solution to be tested are introduced into the measuring cylinder, avoiding foaming as far as possible (if foam has formed on introduction of the formulation solution, the test is not carried out until the foam has disappeared.) The stopper is then put on. The measuring cylinder is shaken vigorously ten times and then put down, starting the stop-clock simultaneously. The height of the foam in the measuring cylinder (volume of the sample including foam) is then read off after predetermined times. The results of the investigation are recorded, stating the sample temperature.

### Example 1

Formulation 1B was investigated as 1.0% strength, 2.0% strength and 3.0% strength solution in the inactivation tests. The exposure times were 5, 15 and 30 minutes. The results are shown in Tables 1-3 below.

TABLE 1
Hepatitis B-inactivating properties of
formulation 1B (1.0%) in the antigen assay. The cpm are
shown.

| Ex- | | | | | | | Inactivation | | |
|---|---|---|---|---|---|---|---|---|---|
| posure | | | | Disinfectant | | | mixture | | |
| time | HBsAg controls | | | controls | | | 1B (1.0%) | | |
| (min) | I | II | III | I | II | III | I | II | III |
| 1 | — | — | — | — | — | — | n.d. | n.d. | n.d. |
| 5 | — | — | — | — | — | — | 1970 | 2256 | 3629 |
| 15 | — | — | — | — | — | — | 1568 | 1781 | 2625 |
| 30 | 7164 | 6609 | 6708 | 125 | 131 | 133 | 1017 | 1560 | 2141 |

n.d. = not done
Diluent control: 186.0
Lower limit of detection of HBsAg in the individual mixtures (cut off)
I: 262.5
II: 275.1
III: 279.3

TABLE 2
Hepatitis B-inactivating properties of
formulation 1B (2.0%) in the antigen assay. The cpm are
shown.

| Ex- | | | | | | | Inactivation | | |
|---|---|---|---|---|---|---|---|---|---|
| posure | | | | Disinfectant | | | mixture | | |
| time | HBsAg controls | | | controls | | | 1B (2.0%) | | |
| (min) | I | II | III | I | II | III | I | II | III |
| 1 | — | — | — | — | — | — | n.d. | n.d. | n.d. |
| 5 | — | — | — | — | — | — | 348 | 448 | 560 |
| 15 | — | — | — | — | — | — | 205 | 238 | 249 |
| 30 | 7164 | 6609 | 6708 | 117 | 115 | 121 | 101 | 105 | 131 |

Diluent control: 186.0
Lower limit of detection of HBsAg in the individual mixtures (cut off)
I: 245.7
II: 241.5
III: 254.1

TABLE 3
Hepatitis B-inactivating properties of
formulation 1B (3.0%) in the antigen assay. The cpm are
shown.

| Ex- | | | | | | | Inactivation | | |
|---|---|---|---|---|---|---|---|---|---|
| posure | | | | Disinfectant | | | mixture | | |
| time | HBsAg controls | | | controls | | | 1B (3.0%) | | |
| (min) | I | II | III | I | II | III | I | II | III |
| 1 | — | — | — | — | — | — | n.d. | n.d. | n.d. |
| 5 | — | — | — | — | — | — | 217 | 339 | 464 |
| 15 | — | — | — | — | — | — | 117 | 127 | 125 |
| 30 | 7164 | 6609 | 6708 | 121 | 110 | 127 | 87 | 99 | 97 |

Diluent control: 186.0
Lower limit of detection of HBsAg in the individual mixtures (cut off)
I: 254.1
II: 231.0
III: 266.7

### Evaluation of Example 1

No interference with the detection method by the presence of disinfectant was evident because the results for the disinfectant controls were in the region of the value for the diluent (cpm=186.0).

Formulation 1B showed a strong effect on the immunological reactivity of HBsAg. However, in the investigation of the 1% strength solution, counts above the lower limit of detection were not seen until after an exposure time of 30 minutes (see Table 1). The 2% strength dilution of the disinfectant in particular showed HBV activity (see Table 2). It can be stated as a result that a 2% strength solution of formulation 1B can be employed for HBV inactivation on use for 15 minutes as surface disinfectant.

According to the Deutsches Ärzteblatt 84, No. 18, page B874 of 30 Apr. 1987, the committee on questions of viral disinfection in human medicine of the German association for the control of viral diseases (DVV) and of the German health agency (BGA) have summed up that all precautionary measures against transmission of hepatitis B are also HIV-preventive.

Exemplary formulation 1B shows that the activity of quaternary ammonium compounds against HBV is increased through use of 1% by weight fatty acid salt (sodium oleate). It was also possible to reduce the total amount of active ingredients.

### Example 2

Formulations 2A and 2B were tested for their HBV activity correspondingly. The results are shown in Table 4 below.

TABLE 4
HBV activity of formulations based on
quaternary ammonium compound

| | |
|---|---|
| 2A | 2B |

| | cut | 15 | 30 | 60 | cut | 15 | 30 | 60 |
|---|---|---|---|---|---|---|---|---|
| | off | min | min | min | off | min | min | min |
| HBV activity | 212.2 | 2020 | 1515 | 1261 | 178.5 | 141 | 92 | 96 |
| (2% strength | | | | | | | | |
| in water) | | | | | | | | |

### Example 3

The procedure was as in Examples 1 and 2, but the suspensions were heated to 55° C.

TABLE 5
HBV activity of formulations based on
alkylamine (at 55° C.)

| | 2A | | | | 2B | | | |
|---|---|---|---|---|---|---|---|---|
| | cut | 15 | 30 | 60 | cut | 15 | 30 | 60 |
| | off | min | min | min | off | min | min | min |
| HBV activity | 333.9 | 7741 | 6798 | 6302 | 312.9 | 593 | 290 | 281 |
| (2% strength | | | | | | | | |
| in water) | | | | | | | | |

It is possible through the use of sodium oleate both to reduce foaming and to improve the action on HBV.

### Example 4 (Reduction in Foaming)

### Formulations Based on Quaternary Ammonium Compound

| | 4A | 4B | 4C |
|---|---|---|---|
| Bardac LF | 15% | 15% | 15% |
| Sodium oleate | | 1% | |
| Sodium citrate | 5% | | |
| Nonionic | 2% | 2% | 2% |
| surfactant | | | |
| Water | 78% | 82% | 83% |

The formulations solutions were 2% strength solutions in tap water at 22° C.

TABLE 6
Foaming of various disinfectant solutions

| | 4A | 4B | 4C |
|---|---|---|---|
| Foaming power, total volume [ml] | 37 | 47 | 72 |
| after 0 s | | | |
| Foaming power, total volume [ml] | 33 | 40 | 70 |
| after 30 s | | | |
| after 1 min | 33 | 38 | 69 |
| after 2 min | 32 | 37 | 67 |
| after 3 min | 32 | 35 | 53 |
| after 4 min | 32 | 35 | 48 |
| after 5 min | 32 | 35 | 45 |

Result:

Formulation 4A showed foam suppression

Formulation 4B showed foam suppression

Formulation 4C showed no foam suppression

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

1-15. (canceled)

16. A composition which may be used as a water-based disinfectant, said composition comprising:
a) a first component, wherein said first component comprises at least one member selected from the group consisting of:
1) an alkylamine; and
2) a quaternary ammonium compound; and
b) a second component, wherein said second component comprises at least one member selected from the group consisting of:
1) a fatty acid RCOOH, wherein R is a group having at least seven carbon atoms; and
2) a salt of said fatty acid RCOOH.

17. The composition of claim 16, wherein said quaternary ammonium compound comprises at least one member selected from the group consisting of:
a) benzalkonium chloride;
b) didecyldimethylammonium chloride; and
c) dioctyldimethylammonium chloride.

18. The composition of claim 16, wherein said alkylamine comprises at least one member selected from the group consisting of:
a) bis(3-aminopropyl)octylamine;
b) aminopropyldodecylamine;
c) dodecylpropylenediamine;
d) coconut fatty amine 2EO; and
e) dimethyl coconut fatty amine.

19. The composition of claim 16, wherein:
a) said fatty acid comprises oleic acid; and
b) said R group comprises an alkyl with a characteristic, wherein:
1) said alkyl comprises a C₉- to C₂₅-alkyl; and
2) said characteristic comprises at least one member selected from the group consisting of:
i) saturated;
ii) unsaturated;
iii) branched; and
iv) unbranched.

20. The composition of claim 19, wherein said alkyl comprises a C₁₁- to C₂₃-alkyl.

21. The composition of claim 20, wherein said alkyl comprises a monounsaturated C₁₃- to C₂₁-alkyl.

22. The composition of claim 16, wherein said salt comprises at least one member selected from the group consisting of:
a) an alkali metal; and
b) ammonium salt.

23. The composition of claim 22, wherein said salt comprises a sodium salt.

24. The composition of claim 23, wherein said salt comprises sodium oleate.

25. The composition of claim 16, wherein said composition comprises said first component in the amount of about 1% to about 40% by weight.

26. The composition of claim 25, wherein said amount is about 3% to about 25% by weight.

27. The composition of claim 26, wherein said amount is about 5% to about 20% by weight.

28. The composition of claim 16, wherein said composition comprises said second component as free acid RCOOH in the amount of about 0.05% to about 5% by weight.

29. The composition of claim 28, wherein said amount is about 0.1% to about 3% by weight.

30. The composition of claim 29, wherein said amount is about 0.5% to about 2% by weight.

31. The composition of claim 28, wherein said composition comprises:
a) said first component in the amount of about 5% to about 20% by weight; and
b) about 0.5% to about 2% by weight of sodium oleate.

32. The composition of claim 31, wherein said first component comprises dioctyidimethylammonium chloride.

33. The composition of claim 16, further comprising at least one nonionic surfactant, short-chain organic acid, wherein said organic acid comprises at least one member selected from the group consisting of:
a) lactic acid;
b) glycolic acid;
c) citric acid;
d) malic acid;
e) succinic acid;
f) tartaric acid;
g) formic acid;
h) acetic acid;
i) propionic acid or salts thereof;
j) corrosion inhibitors;
k) perfume;
l) dye; and
m) alcohols.

34. The composition of claim 33, wherein said composition comprises:
a) said quaternary ammonium compound in the amount of about 5% to about 10% by weight;
b) sodium oleate in the amount of about 0.3% to about 3% by weight; and
c) said nonionic surfactant in the amount of about 0.2% to about 1% by weight.

35. The composition of claim 34, wherein said composition comprises:
a) said quaternary ammonium compound in the amount of about 6% to about 9% by weight;
b) said sodium oleate in the amount of about 0.8% to about 1.6% by weight; and
c) said nonionic surfactant in the amount of about 0.3% to about 0.7% by weight.

36. The composition of claim 35, wherein said quaternary ammonium compound comprises dioctyidimethylammonium chloride.

37. The composition of claim 36, wherein said composition is in the form of a surface disinfectant.

38. The composition of claim 33, wherein said composition comprises:
a) said quaternary ammonium compound in the amount of about 10% to about 20% by weight;
b) sodium oleate in the amount of about 2% to about 7% by weight;
c) said nonionic surfactant in the amount of about 1% to about 3% by weight; and
d) alcohol in the amount of about 10% to about 50% by weight.

39. The composition of claim 38, wherein said composition comprises:
a) said quaternary ammonium compound in the amount of about 13% to about 17% by weight;
b) said sodium oleate in the amount of about 3% to about 5% by weight;
c) said nonionic surfactant in the amount of about 1.5% to about 2.5% by weight; and
d) said alcohol in the amount of about 20% to about 40% by weight.

40. The composition of claim 39, wherein said quaternary ammonium compound comprises dioctyidimethylammonium chloride.

41. The composition of claim 40, wherein said composition is in the form of an instrument disinfectant for thermochemical treatment.

42. A method which may be used for inactivating the hepatitis B virus comprising disinfecting with a composition, wherein said composition comprises:
a) a first component, wherein said first component comprises at least one member selected from the group consisting of:
1) an alkylamine; and
2) a quaternary ammonium compound; and
b) a second component, wherein said second component comprises at least one member selected from the group consisting of:
1) a fatty acid RCOOH, wherein R is a group having at least seven carbon atoms; and
2) a salt of said fatty acid RCOOH.

43. The method of claim 42, further comprising disinfecting surfaces or instruments with said composition.

44. The method of claim 42, further comprising disinfecting with a ready-to-use solution, wherein said solution comprises:
a) water; and
b) said composition in the amount of about 0.3% to about 10% by weight.

45. The method of claim 44, wherein said amount is about 0.5% to about 5% by weight.

46. The method of claim 45, wherein said amount is about 1% to about 3% by weight.

47. A method for disinfecting instruments comprising:
a) pre-cleaning said instruments with cold water;
b) cleaning said instruments with a neutral cleaner, wherein said cleaning occurs at a temperature between about 55° C. to about 60° C.;
c) thermochemically disinfecting said instruments with a disinfectant, wherein:
1) said disinfectant comprises:
i) a first component, wherein said first component comprises at least one member selected from the group consisting of:
aa) an alkylamine; and
bb) a quaternary ammonium compound; and
ii) a second component, wherein said second component comprises at least one member selected from the group consisting of:
aa) a fatty acid RCOOH, wherein R is a group having at least seven carbon atoms; and
bb) a salt of said fatty acid RCOOH;
2) said instruments are exposed to said disinfectant for an exposure time between about 1 minute and 20 minutes; and
3) said thermochemical disinfection occurs at a temperature between about 55° C. to about 60° C.;
d) rinsing said instruments with cold water; and
e) drying said instruments.
